(19) United States
(12) Patent Application Publication
(10) Pub. No.: US 2025/0186034 A1
(43) Pub. Date: Jun. 12, 2025

(54) SURGICAL RETRACTOR

(71) Applicant: Shukla Medical, St. Petersburg, FL (US)

(72) Inventors: Malcolm DEBAUN, Durham, NC (US); Austin Ryan STROH, Lutz, FL (US)

(73) Assignee: Shukla Medical, St. Petersburg, FL (US)

(21) Appl. No.: 18/971,979

(22) Filed: Dec. 6, 2024

Related U.S. Application Data
(60) Provisional application No. 63/606,839, filed on Dec. 6, 2023.

(51) Int. Cl. A61B 17/02; A61B 17/00

(52) U.S. Cl. CPC A61B 17/025; A61B 2017/00367

(57) ABSTRACT

A device for retracting a patient's tissues. More specifically, a surgical retractor is provided which can be easily inserted through an incision in a closed position, and opened to radially actuate a plurality of fingers in contact with the incision to desirably provide 360 degree access to, and visualization of, an operative field. The surgical retractor comprises a housing, a rotatable adjuster mounted to the housing, and a plurality of dilators received in the housing and operatively connected to the rotatable adjuster. Each dilator includes a scroll gear radially movable in the housing, and a finger extending from the scroll gear.

### CROSS-REFERENCE TO RELATED APPLICATION(S)

This application claims the benefit under 35 U.S.C. § 119(e) to U.S. Provisional Application No. 63/606,839, filed Dec. 6, 2023, and entitled “Surgical Retractor,” the disclosure of which is hereby incorporated by reference in its entirety for all purposes.

### FIELD OF THE INVENTION

The present invention relates to a surgical retractor, and more particularly to a surgical retractor that expands in multiple directions.

### BACKGROUND

When performing surgery that requires an incision, a retractor is used to improve access and visibility of the visual field by moving or holding tissue. For example, after an incision is made, a retractor is inserted through the incision and used to keep the skin and/or soft tissue spread open.

### SUMMARY OF THE DISCLOSURE

The present disclosure provides a surgical retractor for retracting a patient's tissues, which provides a surgeon with greater visualization and access to the surgical field. More specifically, a retractor according to the present disclosure can be easily inserted through an incision in its closed position and opened to actuate a plurality of fingers which moves in a radial direction to desirably provide 360 degree access to, and visualization of, an operative field.

According to a first exemplary embodiment of the subject disclosure, a surgical retractor is provided comprising an adjuster, and a plurality of dilators each operatively connected to the adjuster for moving the dilator in a radial direction.

According to an aspect, the surgical retractor further comprises a housing, wherein each of the plurality of dilators is movably mounted to the housing. According to another aspect, the housing includes a plurality of slots each having a dilator slidable mounted thereon. According to another aspect, the housing has an annular body. According to another aspect, the adjuster is mounted to the housing. According to another aspect, the adjuster is a rotatable adjuster. According to another aspect, the adjuster includes a spiral gear for operatively engaging each of the plurality of dilators.

According to an aspect, each of the plurality of dilators includes a scroll gear. According to another aspect, each scroll gear has a transverse cross-section that is substantially T-shaped. According to another aspect, each scroll gear includes a plurality of gear teeth for engaging a spiral gear of the adjuster. According to another aspect, each of the plurality of gear teeth is spaced from a medial end of the scroll gear a distance that differs from a distance each of the plurality of gear teeth is spaced from a medial end of an adjacent scroll gear.

According to an aspect, each of the plurality of dilators includes a depending finger. According to another aspect, each scroll gear includes a finger detachably connected thereto. According to an aspect, each scroll gear includes a female dovetail for receiving a corresponding dovetail joint of a finger therein.

According to an aspect, the surgical retractor comprises a handle connected to the housing.

According to an aspect, a surgical retractor is provided comprising a housing, a rotatable adjuster mounted to the housing, and a plurality of dilators carried by the housing and operatively connected to the rotatable adjuster. Each dilator includes a scroll gear radially movable in the housing, and a finger extending from the scroll gear.

### BRIEF DESCRIPTION OF THE DRAWINGS

The foregoing summary, as well as the following detailed description of the exemplary embodiments, will be better understood when read in conjunction with the appended drawings. For the purpose of illustration, there are shown in the drawings exemplary embodiments of the subject disclosure. It should be understood, however, that the subject disclosure is not limited to the precise arrangements and instrumentalities shown.

FIG. 1 is a perspective view of a first exemplary embodiment of a surgical retractor of the subject disclosure;

FIG. 2 is an exploded view of the surgical retractor of FIG. 1;

FIGS. 3A-3C are a top perspective view, a bottom perspective view, and a cross-sectional view, respectively, of an exemplary adjuster of the surgical retractor of FIG. 1;

FIGS. 4A and 4B are a bottom perspective view and a top perspective view, respectively, of an exemplary housing of the surgical retractor of FIG. 1;

FIG. 5 is a perspective view of an exemplary retaining ring of the surgical retractor of FIG. 1;

FIGS. 6A and 6B are a perspective view (A) and a top view (B) of an exemplary scroll gear of the surgical retractor of FIG. 1;

FIG. 7 is a perspective view of an exemplary finger of the surgical retractor of FIG. 1;

FIG. 8 is a perspective view of an exemplary handle of the surgical retractor of FIG. 1;

FIGS. 9A and 9B are various top views of the surgical retractor of FIG. 1 in a closed position (A) and an open position (B);

FIG. 10 is a perspective view of an exemplary housing and dilators of the surgical retractor of FIG. 1;

FIG. 11 is a perspective view of a second exemplary embodiment of a surgical retractor of the subject disclosure;

FIG. 12 is a top perspective view of an exemplary housing of the surgical extractor of FIG. 11;

FIGS. 13A-13C are various views of an exemplary scroll gear of the surgical extractor of FIG. 11;

FIGS. 14A-14C are various views of an exemplary finger of the surgical extractor of FIG. 11;

FIG. 15 is a side view of an exemplary threaded fastener of the surgical extractor of FIG. 11;

FIGS. 16A and 16B are a perspective view and a top plan view, respectively, of an exemplary handle of the surgical extractor of FIG. 11; and

FIGS. 17A and 17B are various views of an exemplary handle release button of the surgical extractor of FIG. 11.

### DETAILED DESCRIPTION

Reference will now be made in detail to various examples of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout this disclosure, various aspects of the subject disclosure can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages, and characteristics of the exemplary embodiments of the subject disclosure may be combined in any suitable manner in one or more embodiments. One skilled in the relevant art will recognize, in light of the description herein, that the present disclosure can be practiced without one or more of the specific features or advantages of a particular exemplary embodiment. In other instances, additional features and advantages may be recognized in certain embodiments that may not be present in all exemplary embodiments of the subject disclosure.

The subject disclosure provides a surgical retractor that requires minimal space for insertion in its closed position and, in its open position, retracts a patient's tissues in multidirectional manner e.g. via a plurality of fingers that move in a radial direction to provide a surgeon with improved access and visualization of the operative field compared to prior art retractors.

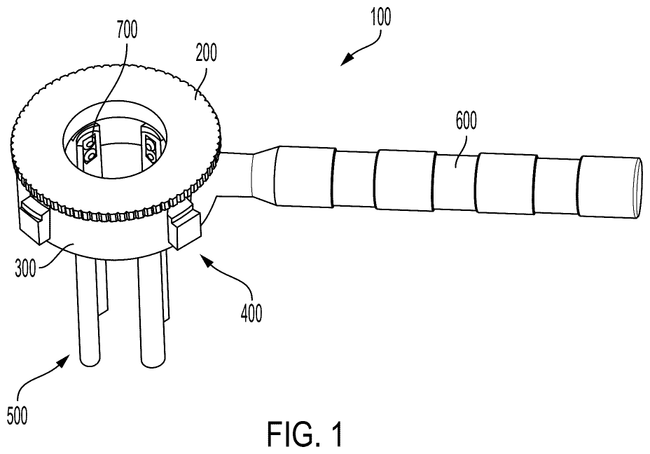

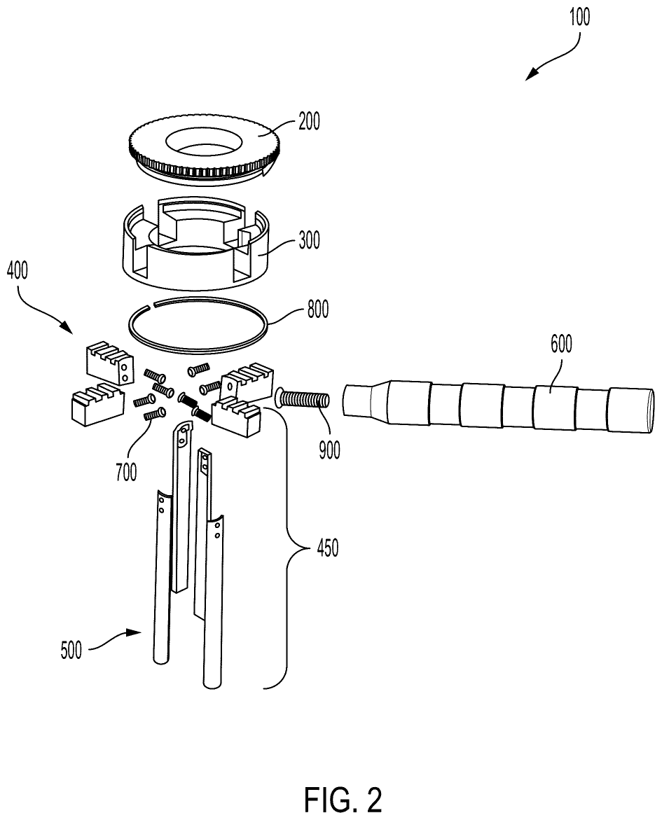

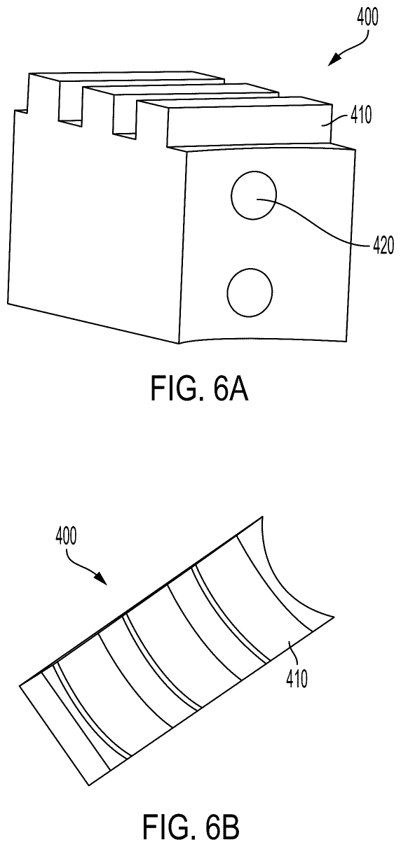

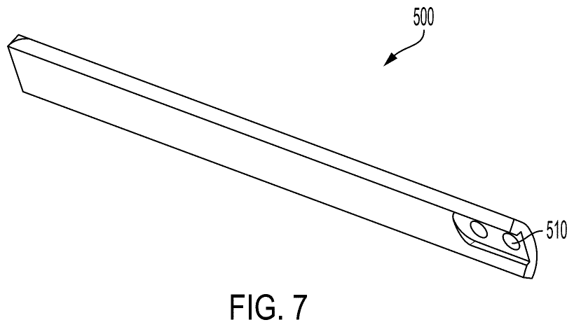

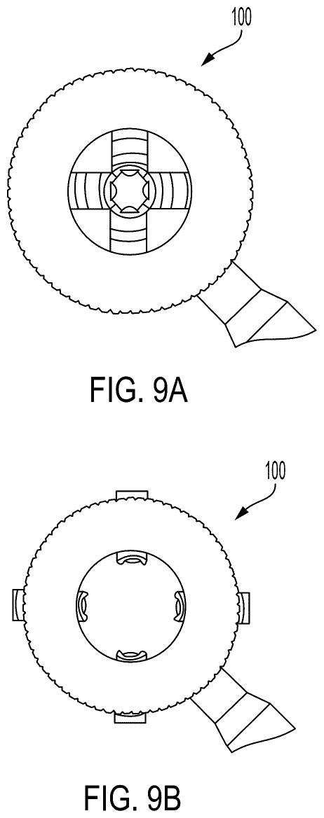

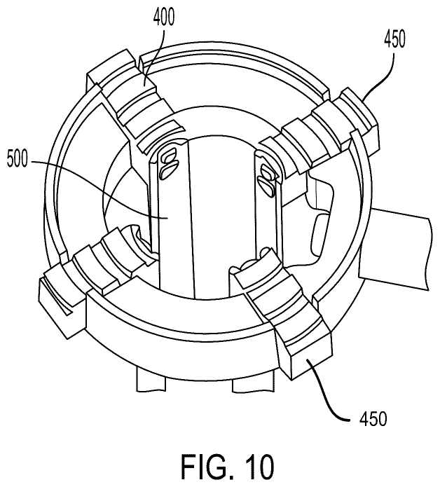

In accordance with a first exemplary embodiment, the present disclosure provides for a surgical retractor 100, as best shown in FIGS. 1-10. The surgical retractor 100 includes an adjustment mechanism and a plurality of dilators 450, each of the plurality of dilators 450 having of a scroll gear 400 and a finger 500 (FIG. 2). Each of the plurality of dilators is movable in a radial direction. For example, each of the plurality of dilators is movable in a radial direction toward and away from a rotational axis or central axis defined by a circle based upon the positions of each of the fingers. The surgical retractor further includes a housing 300 for housing the adjustment mechanism and the plurality of dilators, or wherein the adjustment mechanism and dilators are mounted. According to an exemplary embodiment, the surgical retractor 100 includes at least two or, more preferably, three or more dilators 450.

The adjustment mechanism is configured as best shown in FIGS. 1-3C. The adjustment mechanism can be configured as an adjuster or rotatable adjuster 200. The adjuster 200 includes a circular plate 202 having a textured outer rim 204 for enhancing a user's grip and a spiral gear 206 on a bottom face of the circular plate, and a planar top face. The circular plate 202 also includes a central through hole 208. Adjacent the spiral gear, the adjuster includes an annular recess 209.

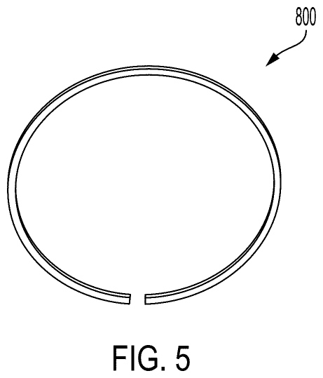

The housing 300 is configured as best shown in FIGS. 1, 2, 4A and 4B. The housing 300 has a substantially annular body. The upper, inner circumference of the housing 300 includes an annular recess 302 for receiving a locking retaining ring 800 therein (FIGS. 2 and 5). The housing also includes a plurality of slots 310, each configured to receive a respective dilator therein. The housing can include any number of slots to match or correspond to an equivalent number of dilators configured for the surgical retractor, e.g., 2, 3, 4, 5, 6, 7, 8, 9, and 10 slots. The slots 310 are circumferentially spaced apart from each other and are substantially rectangularly shaped in elevational profile (e.g., having a substantially rectangularly shaped longitudinal cross-section) with an open proximal (superior) side and a planar and smooth bottom surface to promote sliding of a dilator thereon. The housing 300 also includes a through hole 320 in its lateral aspect to receive and fasten to a handle 600. The through hole 320 can be configured, e.g., as a threaded through hole.

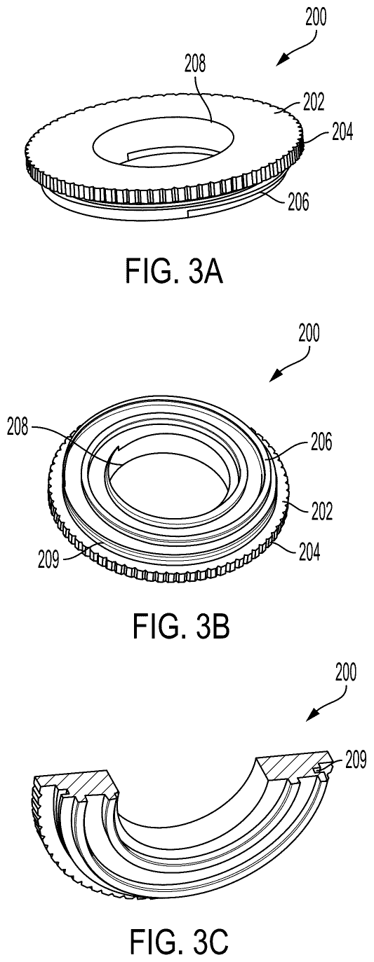

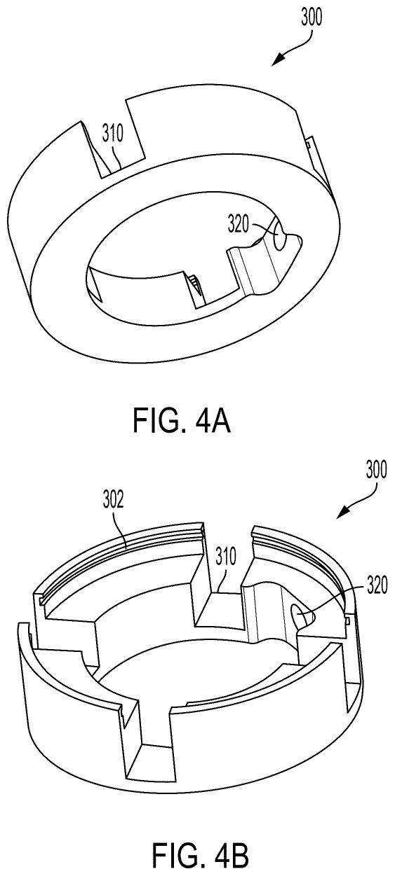

The locking retaining ring 800 is configured as best shown in FIGS. 2 and 5. The retaining ring 800 is configured to fit within the annular recess 302 of the housing 300 (FIG. 4B) and the corresponding annular recess 209 of the adjuster 200 (FIGS. 3b and 3C) to lock the adjuster to the housing.

The scroll gear 400 is configured as best shown in FIGS. 1, 2, 6A and 6B. The scroll gear 400 is substantially rectangular with a concave interior surface (i.e., concave medial surface), and is configured to slide within the slot 310 of the housing. The superior or top surface of the scroll gear includes a plurality, e.g., three curved gear teeth or ridges 410 configured to operatively engage with the spiral gear of the adjustment mechanism. Alternatively, the scroll gear 400 can also include more or less gear teeth corresponding to the number of spiral gear revolutions provided on the adjuster. For example, the adjuster can include a spiral gear with 2, 3, 4, 5, 6, 7, 8, 9 or 10 revolutions; and the scroll gear 400 can correspondingly include 2, 3, 4, 5, 6, 7, 8, 9 or 10 corresponding gear teeth or ridges 410. Moreover, each of the at least two gear teeth is spaced from a medial end of the scroll gear (i.e., the concave interior surface) a distance that differs from a distance each of the at least two gear teeth is spaced from a medial end of an adjacent scroll gear.

The inner surface or medial face of the scroll gear 400 includes recessed holes 420, e.g., threaded counterbores, configured to receive fasteners e.g., screws 700 (FIG. 2) that attach the finger 500 to the scroll gear.

The scroll gear 400 is self-locking to maintain the fingers 500 in a desired location. The scroll gear 400 is self-locking via a small angle at which the gear teeth contacts the revolutions of the spiral gear 206. When the scroll gear 400 pushes back on the adjuster 200 there is not enough force to overcome inherent friction in the assembly. Due to the inherent friction of the assembly, the scroll gear 400 cannot backdrive the adjuster 200 thereby rendering the assembly self-locking.

The finger 500 is configured as best shown in FIGS. 1, 2 and 7. The finger 500 is an elongated structure with holes about its proximal end configured to receive fasteners 700 that attach the finger to the scroll gear 400. The holes can be recessed to accommodate the screw heads. The distal end of the finger 500 is rounded to minimize trauma to the patient's tissues upon insertion of the finger into an incision. The finger 500 can be any length suitable for its intended purpose. For example, the finger 500 can be 2, 3, 4, 5, 6, 7, or 8+ inches long. Each of the plurality of fingers 500 of the surgical retractor may be the same length or different lengths.

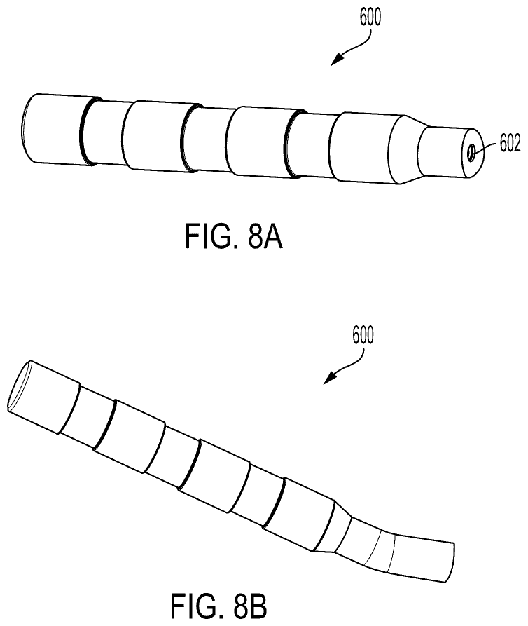

A handle 600 according to an exemplary first embodiment of the subject disclosure is configured as best shown in FIGS. 1, 2 and 8. The handle 600 is elongated and substantially cylindrical. A medial end of the handle 600 includes a threaded hole 602. A fastener e.g., a screw 900 is inserted through a corresponding hole in the housing 320 and threaded into the threaded hole 602 in the handle 600, thereby securing the handle to the housing 300. The handle 600 can be comprised of telescoped segments 604, which permits the handle to bend (FIG. 8B) to better conform to a patient's anatomy or user requirements.

It will be understood that sufficient rotation of the adjuster 200 by a user's hand in a first direction causes the dilators 450 to assume a radially innermost position whereby the fingers 500 of the dilators are closest together for insertion into a surgical incision. Once the fingers 500 are received in the incision, the rotatable adjuster 200 is rotated in a second direction opposite the first direction to cause the dilators 450 and their fingers 500 to assume radially expanded positions, thereby separating the patient's tissues in multiple directions to desirably provide improved access to, and visualization of, an operative field.

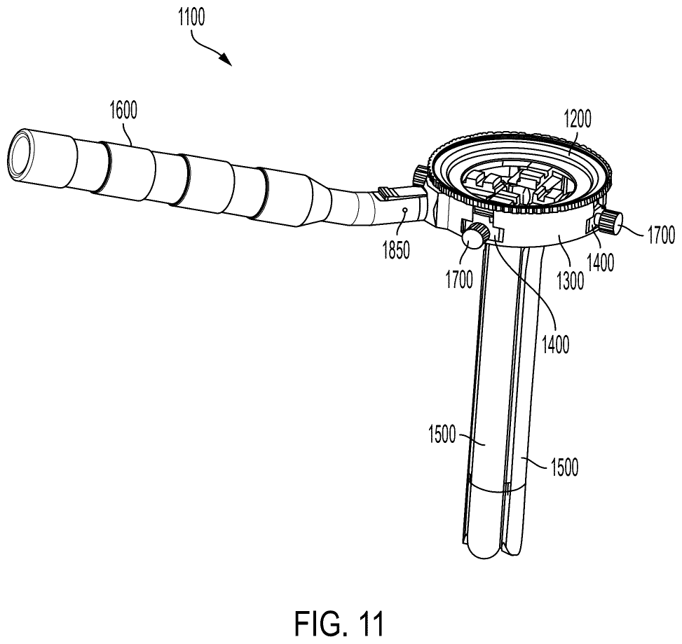

In accordance with a second exemplary embodiment, the present disclosure provides for a surgical retractor 1100, as best shown in FIG. 11. The surgical retractor 1100 is similar in construction and function to the surgical retractor 100 described above. Accordingly, only those features of the surgical retractor 1100 which depart materially in structure and/or function from their counterparts described above will be described in detail herein. The surgical retractor 1100 comprises an adjustment mechanism configured as a rotatable adjuster or adjuster 1200 which is constructed and functions substantially similar to the adjuster 200 described above. In addition, the surgical retractor 1100 includes a housing 1300, a plurality of scroll gears 1400, a plurality of fingers 1500 (wherein a scroll gear and a finger together define a dilator), a handle 1600, and a plurality of fasteners 1700, all of which will be described in greater detail below.

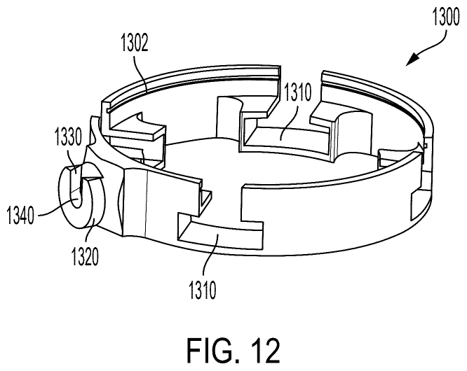

The housing 1300 is configured as best shown in FIG. 12. The housing 1300 is a substantially annular structure. The upper, inner circumference of the housing 1300 includes an annular recess 1302 for receiving an unillustrated retaining ring therein (similar to retaining ring 800 described above) which engages a corresponding slot in the adjuster 1200 to retain the adjuster in the housing 1300. The housing also includes a plurality of slots 1310, each configured to receive a respective dilator therein. The housing can include any number of slots to match or correspond to an equivalent number of dilators configured for the surgical retractor, e.g., 2, 3, 4, 5, 6, 7, 8, 9, and 10 slots. The slots 1310 are circumferentially spaced apart from each other and have a transverse cross-section substantially T-shaped with an open proximal, medial and superior side and a planar bottom surface to promote sliding of the dilator thereon. The housing 1300 also includes a handle fastener 1320 about its lateral aspect to receive and fasten to the handle 1600.

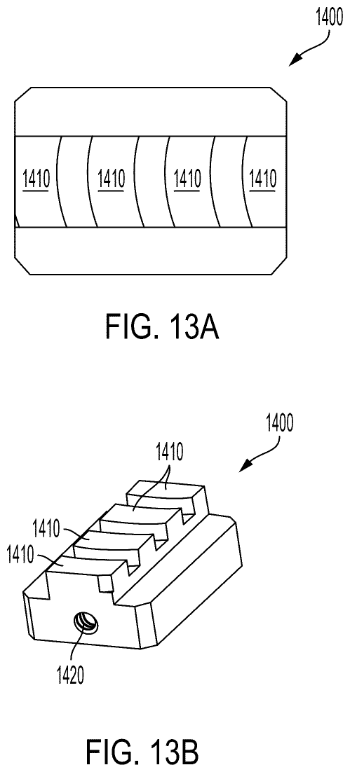

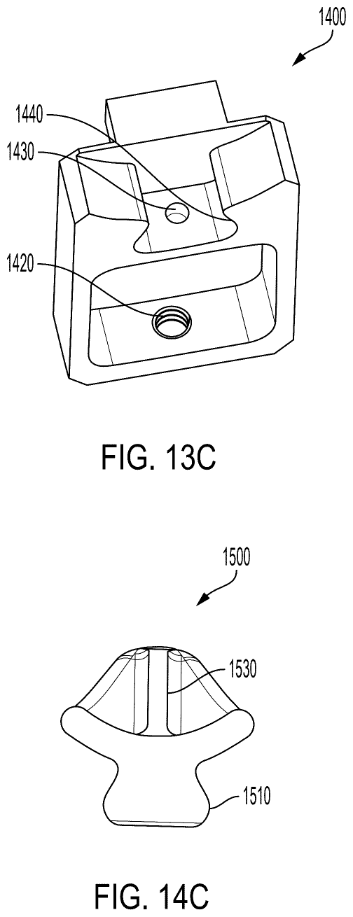

The scroll gear 1400 is configured as best shown in FIGS. 13A-13C. The scroll gear 1400 has a transverse cross-section substantially T-shaped and configured to slide within the slot 1310 of the housing 1300. The superior or top surface of the scroll gear includes a plurality, e.g., four curved gear teeth or ridges 1410 configured to operatively engage with the spiral gear of the adjuster. Alternatively, the scroll gear 400 can also include more or less gear teeth corresponding to the number of spiral gear revolutions provided on the adjustment mechanism. For example, the adjustment mechanism can include a spiral gear with 2, 3, 4, 5, 6, 7, 8, 9 or 10 revolutions; and the scroll gear 1400 can, correspondingly, include 2, 3, 4, 5, 6, 7, 8, 9 or 10 corresponding gear teeth or ridges 1410. Moreover, each of the at least two gear teeth 1410 is spaced from a medial end or medal face of the scroll gear a distance that differs from a distance each of the at least two gear teeth is spaced from a medial end or medial face of an adjacent scroll gear.

As shown in FIGS. 13B and 13C, a lateral end of the scroll gear 1400 further includes a threaded opening 1420 for receiving a threaded portion of the fastener 1700. Additionally, as shown in FIG. 13C, the scroll gear 1400 includes a through hole 1430 in alignment with the threaded opening 1420 and in communication with a female dovetail recess 1440, the functions of which will be described in detail below.

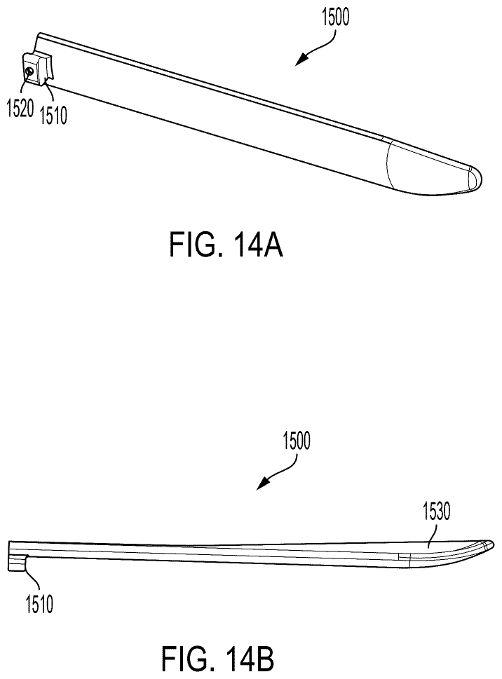

FIGS. 14A-14C illustrate a finger 1500 constructed in accordance with the second exemplary embodiment of the subject disclosure. The finger 1500 is an elongated structure having a male dovetail joint 1510 adjacent its proximal end which is configured to matingly engage with the female dovetail recess 1440 provided in the scroll gear 1400. The male dovetail joint 1510 includes a hole or recess 1520 which is configured to align with the through hole 1430 provided adjacent the female dovetail formation 1440 of the scroll gear 1400 and with the threaded opening 1420 when the male dovetail joint 1510 is received in the female dovetail recess 1440. The distal end of the finger 1500 is rounded to minimize trauma to the patient's tissues upon insertion of the finger into an incision. The finger 1500 also preferably includes an elongated stiffening rib 1530 to resist bending of the finger as bodily tissues are retracted by the surgical retractor 1100. The finger 1500 can be any length suitable for its intended purpose. For example, the finger 1500 can be 2, 3, 4, 5, 6, 7, or 8+ inches long. Each of the plurality of fingers 1500 of the surgical retractor may be the same length or different lengths.

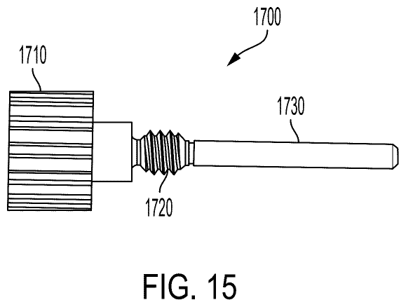

FIG. 15 shows an exemplary embodiment of the fastener 1700 for releasably attaching the finger 1500 to the scroll gear 1400. The fastener 1700 includes an enlarged wheel or a thumb wheel 1710 about one end thereof for facilitating gripping and turning of the fastener and threadingly engaging the fastener with the scroll gear 1400. Adjacent the enlarged wheel 1710, the fastener includes a threaded portion 1720 having threads configured to threadedly engage the threaded opening 1420 of the scroll gear 1400. At its distal end the threaded fastener includes an elongated pin 1730 which is configured to be received in the alignable holes of the female dovetail recess of the scroll gear 1400 and the male dovetail joint of the finger 1500. More particularly, when the male dovetail joint 1510 of the finger is received in the female dovetail recess 1440 of the scroll gear, the threaded opening 1420, the through hole 1430 and the hole 1520 are coaxially aligned. Once the threaded opening 1420 and the holes 1430 and 1520 are aligned, a user turns the enlarged wheel 1710 in a first direction whereby the threaded portion 1720 of fastener 1700 is threaded into the threaded opening 1420 such that the elongated pin 1730 is inserted into the aligned holes 1430, 1520 to attach the finger 1500 to the scroll gear 1400 to form a dilator. When it is desired to detach the finger from the scroll gear, a user turns the enlarged wheel 1710 in a second direction opposite the first direction, thereby unthreading the threaded portion 1720 from the threaded opening 1420 such that the elongated pin 1730 is withdrawn from the holes 1430 and 1520. With the elongated pin 1730 withdrawn from the holes 1430 and 1520, a user may simply pull down on the finger 1500 to free the male dovetail joint 1510 of the finger from the female dovetail recess 1440 of the scroll gear and detach the finger from the scroll gear.

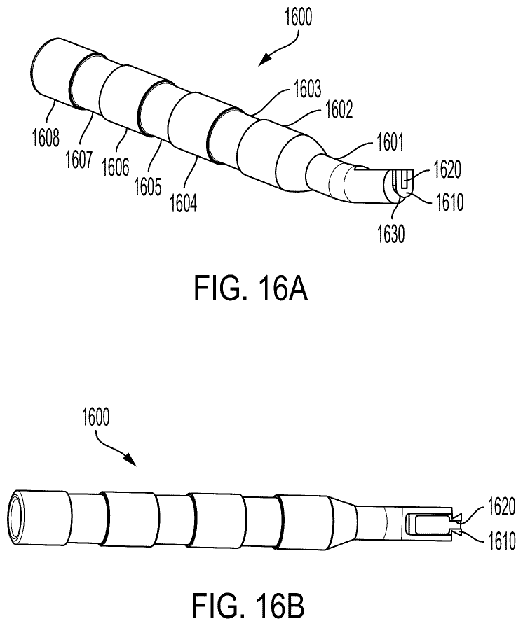

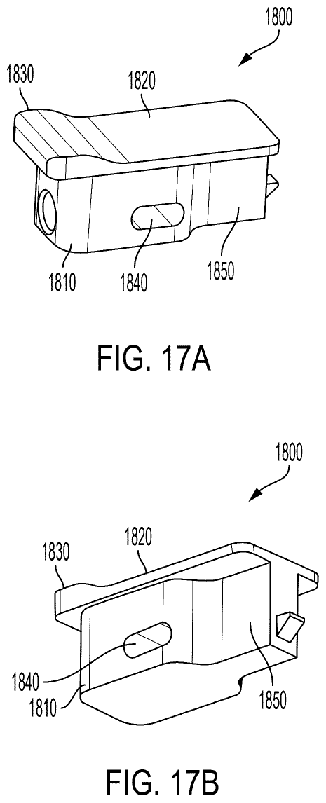

A handle 1600 according to the exemplary second embodiment of the subject disclosure is configured as best shown in FIGS. 16A and 16B. The handle 1600 is elongated and substantially cylindrical. The handle can be comprised of telescoped segments 1601-1608, which permit the handle to be bent based on a patient's anatomy. A distal end of the handle 1600 includes a male dovetail 1610 having a slot 1620. The male dovetail 1610 terminates at a rounded stop portion 1630 about its lower end. The male dovetail 1610 is configured to be selectively inserted into and withdrawn from a female dovetail pocket 1330 provided in the handle fastener 1320 of the housing 1300 (FIG. 12) and releasably retained therein by a handle release button 1800 (FIGS. 17A and 17B), as described below. The female dovetail pocket 1330 includes a slot 1340 configured to align with the slot 1620 of the male dovetail 1610 of the handle 1600.

Referring the FIGS. 17A, 17B, the handle release button 1800 is shown to comprise a lower body portion 1810 and an upper body portion 1820 having a raised formation 1830 adapted to be moved forwardly and rearwardly by a user's thumb. The lower body portion 1810 includes an elongated slot 1840 for receiving a pin 1850 (FIG. 11) which slidably connects the handle release button 1800 to the handle 1600. The elongated slot 1840 of the lower body portion also includes a relatively narrow region 1850 (e.g., a region having a width less than a width of the lower body portion 1810) configured to be inserted into and withdrawn from the aligned slots 1340, 1620 when the male dovetail 1610 of the handle 1600 is received in the female dovetail pocket 1330 of the housing 1300. More particularly, using his or her thumb to move the raised formation 1830, the user selectively pushes the handle release button 1800 forwardly to drive the relatively narrow region 1850 into the aligned slots 1340, 1840 to secure the handle 1600 to the housing 1300. When it is desired to remove the handle, the user moves the raised formation 1830 and thus the handle release button rearwardly, thereby withdrawing the relatively narrow region 1850 from the aligned slots 1340, 1840 whereby the handle may be lifted from engagement from the housing.

It will be appreciated by those skilled in the art that changes could be made to the exemplary embodiments described above without departing from the broad inventive concept thereof. It is to be understood, therefore, that this disclosure is not limited to the particular embodiments disclosed, but it is intended to cover modifications within the spirit and scope of the subject disclosure as defined by the appended claims.

1. A surgical retractor comprising:
an adjuster; and
a plurality of dilators each operatively connected to the adjuster for moving the dilator in a radial direction.

2. The surgical retractor of claim 1, further comprising a housing, wherein each of the plurality of dilators is movably mounted to the housing.

3. The surgical retractor of claim 2, wherein the housing includes a plurality of slots, each having a dilator slidably mounted therein.

4. The surgical retractor of claim 2, wherein the housing has an annular body.

5. The surgical retractor of claim 2, wherein the adjuster is mounted to the housing.

6. The surgical retractor of claim 1, wherein the adjuster is a rotatable adjuster.

7. The surgical retractor of claim 1, wherein the adjuster includes a spiral gear for operatively engaging each of the plurality of dilators.

8. The surgical retractor of claim 1, wherein each of the plurality of dilators includes a scroll gear.

9. The surgical retractor of claim 8, wherein each scroll gear has a transverse cross-section that is substantially T-shaped.

10. The surgical retractor of claim 8, wherein each scroll gear includes a plurality of gear teeth for engaging a spiral gear of the adjuster.

11. The surgical retractor of claim 10, wherein each of the plurality of gear teeth is spaced from a medial end of the scroll gear a distance that differs from a distance each of the plurality of gear teeth is spaced from a medial end of an adjacent scroll gear.

12. The surgical retractor of claim 1, wherein each of the plurality of dilators includes a depending finger.

13. The surgical retractor of claim 8, wherein each scroll gear includes a finger detachably connected thereto.

14. The surgical retractor of claim 8, wherein each scroll gear includes a female dovetail for receiving a corresponding dovetail joint of a finger therein.

15. The surgical retractor of claim 2, further comprising a handle connected to the housing.

16. A surgical retractor comprising:
a housing;
an rotatable adjuster mounted to the housing; and
a plurality of dilators carried by the housing and operatively connected to the rotatable adjuster; each of the plurality of dilators including:
a scroll gear radially movable in the housing, and
a finger extending from the scroll gear.
